(19) United States
(12) Patent Application Publication
(10) Pub. No.: US 2008/0213899 A1
(43) Pub. Date: Sep. 4, 2008

(54) Rotationally Oscillating Injector

(75) Inventor: Nejat Olgac, Coventry, CT (US)

Correspondence Address: MCCARTER & ENGLISH , LLP STAMFORD OFFICE, FINANCIAL CENTRE , SUITE 304A, 695 EAST MAIN STREET, STAMFORD, CT, 06901-2138, US

(73) Assignee: UNIVERSITY OF CONNECTICUT, Farmington, CT (US)

(21) Appl. No.: 11/871,760

(22) Filed: Oct. 12, 2007

Related U.S. Application Data
(60) Provisional application No. 60/851,348, filed on Oct. 12, 2006.

(51) Int. Cl. C12N 15/87; B01J 19/00; G01N 1/00; C12M 1/00

(52) U.S. Cl. 435/455; 422/99; 435/285.1; 73/863

(57) ABSTRACT

A microinjection device is provided that includes an injection element defining a longitudinal axis, and that further includes a motor. The injection element is rotatable about the longitudinal axis by the rotational motor. The injection element is for penetrating a target, such as a cell. A microinjection system is provided that includes the microinjection device and a control unit. The control unit is for controlling a rotational amplitude and a frequency of oscillation of the injection element. A method for penetrating a target to facilitate injecting material therein is provided that includes providing the material to an injection element, contacting the target with a distal end of the injection element, rotating the injection element about a longitudinal axis to form a hole in the target, and penetrating the target with the injection element via the hole formed in the target. A method for performing intra-cytoplasmic sperm injection is provided that includes providing a solution comprising sperm to an injection element, contacting an oocyte with a distal end of the injection element, rotating the injection element alternately clockwise and counterclockwise about a longitudinal axis to form a hole in the oocyte, penetrating the oocyte with the distal end of the injection element via the hole formed in the oocyte, and expelling the solution comprising sperm into the penetrated oocyte.

### CROSS-REFERENCE TO RELATED APPLICATION

This application claims priority from the United States Provisional Patent Application entitled “Rotationally Oscillating Microinjector” having Ser. No. 60/851,348 and filed on Oct. 12, 2006, the disclosure of which is incorporated herein by reference.

### FEDERALLY SPONSORED RESEARCH

The work described in this application was sponsored in part by the National Institutes of Health (NIH), a part of the United States Department of Health and Human Services.

### BACKGROUND

1. Technical Field

The present disclosure is directed to devices, systems and methods for injection. More particularly, the present disclosure is directed to devices, systems, and methods for biological microinjection.

2. Background Art

Many procedures involve injecting genetic and other material into biological cells and nuclei. Cloning, in-vitro fertilization, genetic research, and disease therapies all involve injections, and typically are done by a micropipette, guided by a micromanipulator, to penetrate the cell wall and in some cases, to enter the cell nucleus.

Many procedures involve injecting genetic and other material into biological cells and nuclei, often involving the use of a micropipette guided by a micromanipulator to penetrate the respective cell wall and/or to enter the cell nucleus. For example, FIG. 1 shows a pre-penetration stage of a microinjection procedure, wherein a cell 100 is stabilized in place by a holding pipette 102, and a micropipette 104 is positioned outside the cell zona 106. During the microinjection procedure, the micropipette 104 is inserted through the zona pellucida or cell zona 106 and the oolemma or cell membrane 108 and the contents of the pipette are expelled into the inside of the cell. For example, during in-vitro fertilization procedures, sperm may be injected via a micropipette into an oocyte.

In recent years, cellular piercing has become a crucial procedure in cellular biology, especially in various nuclear or subcellular transfer operations, in-vitro fertilization, genetic research (e.g., involving DNA microinjection) and disease therapies. Information regarding the proliferation of cellular piercing procedures is set forth in Spring-Verlag Berlin Heidelberg, pp. 23-40 (2006) by M. W. Li and K. C. K. Lloyd (Li 2006); and in Reprod. Biomed. Online, Vol. 10, pp. 247-88 (2005) by R. Yanagimachi (Yanagimachi 2005). One such procedure, intracytoplasmic sperm injection (ICSI) has been successfully used on a variety of species, including mouse, rat, and cattle. Examples of such procedures are set forth in Zygote, Vol. 6, No. 2 (1998) by D. Dozortsev et al. (Dozortsev 1998); Reprod. Biomed. Online, Vol. 5, No. 3, pp. 270-272 (2002) by A. Fonttis et al. (Fonttis 2002); Hum. Reprod., Vol. 12, pp. 1062-1068 (1997) by L. Meng and D. P. Wolf (Meng 1997); Theriogenology, Vol. 54, No. 6, pp. 935-948 (2000) by R. Suttner et al. (Suttner 2000); and Nat. Biotechnol., Vol. 16, pp. 639-641 (July 1998) by T. Wakayama et al. (Wakayama 1998). As set forth in the following references, ICSI has also used very effectively in treating male factor infertility: Hum. Reprod., Vol. 17, No. 3, pp. 671-694 (2002) by M. Bonduelle et al. (Bonduelle 2002); Fonttis 2002; Lancet Vol. 340 (1992) by G. Palermo et al. (Palermo 1992); Hum Reprod., Vol. 17, No. 2, pp. 362-369 (2002) by M. Plachot et al. (Plachot 2002); Gynecol. Obstet. Investig., Vol. 52, pp. 158-162 (2001) by S. Takeuchi et al. (Takeuchi 2001); and Hum. Reprod., Vol. 14, No. 2, p.p. 448-453 (1998) by K. Yanagida et al. (Yanagida 1998).

Many methods and instruments may be used to facilitate cell wall puncture. In some early applications of ICSI, commonly referred to as “Conventional ICSI”, injections were conducted manually. Some such early ICSI applications are described in Mol. Reprod. Dev., Vol. 38, No. 3, pp. 264-267 (1994) by P. Collas et al. (Collas 1994); Meng 1997; and Takeuchi 2001. In accordance with such techniques, the spiked tip of an injection pipette is pushed gently about halfway through the oocyte, after which the injection pipette is pushed forward swiftly to penetrate the zona pellucida and the membrane or oolemma. Once the pipette tip is inside the cell, the whole sperm is injected into the ooplasm.

Conventional ICSI in most of the species, including cattle, rat, and mouse, have proven mostly unsuccessful. For example, in the first instance, the very elastic oolemma in the mouse is very difficult to penetrate, and the length of the mouse sperm tail that results in excessive deposition of medium when injecting the whole, intact sperm, makes convention ICSI extremely difficult to perform successfully in the mouse. Some such difficulties are reported in Li 2006; Hum. Reprod., Vol. 10, pp. 2831-2834 (1995) by R. Ron-El et al. (Ron-El 1995); Hum. Reprod., Vol. 10, pp. 2642-2649 (1995) by O. Lacham-Kaplan and A. Trounson (Lacham-Kaplan 1995); and Hum. Reprod., Vol. 10, pp. 431-435 (1995) by A. Ahmadi et al. (Ahmadi 1995).

A failure rate of greater than 90% for conventional ICSI was indicated in Collas 1994. Such failures are commonly attributed to damage to the membrane or the zona and/or cell deformation occurring during the piercing process, inhibiting cell viability, and/or rendering the cell unusable for a particular task. To the extent such damage does not heal effectively, an abnormal growth may occur in the future stages of development. Accordingly, the particular techniques used for cellular piercing and ensuing microinjection can be vitally important for the success of the overall ICSI procedure.

An enhanced version of the ICSI process, known as “piezo-assisted ICSI” or “piezo-assisted cellular piercing” has been proven to improve the success rate beyond the conventional ICSI. Examples of piezo-assisted ICSI are set forth in Assist. Reprod. Genet., Vol. 13, No. 4, 320-328 (1996) by T. Huang et al. (Huang 1996); Theriogeneology, Vol. 52, 1215-1224 (1999) by H. Katayose et al. (Katayose 1999); Biol. Reproduct., Vol. 52, No. 4, 709-720 (1995) by Y Kimura et al. (Kimura 1995); Fertil. Steril., Vol. 69, No. 4 (1998) by T. Nakayama et al. (Nakayama 1998); and Takeuchi 2001. The cellular piercing may be done using two glass pipettes (Dozortsev 1998, Fonttis 2002), which may be referred to as the holding and the injection pipettes. They vary in size and shape depending on the species of the cell and the physical characteristic of the membrane. For example, for ICSI on mice oocytes, the injection pipette may have a tip with an outer diameter of about 7 μm and an inner diameter of about 5 μm. The holding pipette may have a tip having an outer diameter of about 50-100 μm and an inner diameter of about 10 μm, and may be used to immobilize the oocyte by a slight suction. Referring now to FIG. 2, showing a cell 200 having a cell zona 202 and a cell membrane 204, and showing an injection pipette 206 defining a longitudinal axis 208, the injection pipette 206 is pressed on the cell membrane 204 and a dimple is generated. A commercially-available piezo-electrically actuated impact type force generator (not shown), such as the Piezo-Drill of Burleigh Instruments, may be used to oscillate the injection pipette 206 along the longitudinal axis 208, typically on the order of nanometers, to facilitate penetrating the cell zona 202 and the cell membrane 204.

Piezo-assisted cellular piercing procedures represent important progress toward automated and repeatable deployment of microinjection operation. However, a number of points in the physics of such procedures may be of interest in terms of further investigation. In this regard, ICSI on mouse oocytes is of particular interest to cell biologists as such oocytes are used very broadly for pharmaceutical purposes.

As demonstrated by Ediz and Olgac in “Microdynamics of the Piezo-Driven Pipettes in ICSI”, IEEE Transactions on Biomedical Engineering, Vol. 51, No. 7, p. 1262, July 2004, and by Ediz and Olgac in “Effects of Mercury Column on the Microdynamics of the Piezo-Driven Pipettes”, ASME Journal of Biomechanical Engineering, Vol. 127, pp. 531-535, 2002, each of which is incorporated herein by reference in its entirety, piezo-actuated impulsive axial forcing generates transverse displacements at the tip of the micropipette that are more intensive than the intended overall axial or longitudinal motion of the micropipette. These movements are transmitted and further exaggerated through the flexible micropipette tip. As shown in FIGS. 3 and 4, respectively, such transverse motion of the pipette tip may be observed via microscopic high speed photography in air and cell medium. More particularly, FIGS. 3 and 4 track the transverse motion of the pipette tip as a result of an oscillation of the pipette in the longitudinal direction. In such circumstances, the amplitude of motion of the pipette tip in the transverse dimension can easily be an order of magnitude greater than the overall amplitude of motion of the pipette in the longitudinal direction. This relatively large motion of the pipette tip in the transverse direction may contribute to the damage of the cellular structure upon injection.

Several groups working in the piezo-assisted ICSI field confirm an interesting outcome in which a very small mercury column used in the pipette can improve the success rate by dampening such undesirable oscillations in the transverse dimension. Examples of such reports are set forth in Biol. Reprod., Vol. 66, 381-385 (2002) by Y. Kawase et al. (Kawase 2002), Kimura 1995, Biol. Reprod., Vol. 58, No. 6, 1407-1415 (1998) by Y. Kimura et al. (Kimura 1998); and Hum. Reprod., Vol. 14, No. 2, 448-453 (1998) by K. Yanagida et al. (Yanagida 1998). For successful piercing, a small amount of mercury (approximately 0.5 μl) may be placed at the tip of the injection pipette from the proximal end. After the mercury column is pushed close to the tip, a minute amount of medium is sucked from the front end. The injection pipette is connected to the pipette holder, which is attached to a syringe system filled with mineral oil. The pipette holder is, in turn, connected to the piezo-drill. Referring now to FIG. 5, showing a holding pipette 500, an injection pipette 502, and a dish 504, during the ICSI process, the tip of each of the holding and injection pipettes 500, 502 are dipped in a droplet 506 of medium, which is highly viscous and contains the oocyte 508 and the sperms inside. This high-viscosity medium is used to immobilize the sperms in the drop. On top of them, the whole dish 504 is filled with mineral oil 510 covering the drop and the drawn sections of the pipettes. In this configuration, the holding pipette 500 clamps the oocyte via suction, while the injection pipette 502 is touched gently to the zona of the oocyte 508.

The most common problem is damage to the membrane of the oocyte 508 caused by the piezo pulse. If these pulses damage the cell membrane, the cytoplasm leaks out and the oocyte 508 lyses in a few seconds. To prevent such occurrences the controller parameters of the piezo drill are set a comfortable operating conditions, which will encompass the amplitude, duration, and the frequency of impulse force train. The conditions that are effective for the mouse will generally vary from those that are effective for other species. These variations and selections of the most effective settings require a great deal of operator expertise and training. Such requirements impose severe restrictions on the day-to-day conduct in the laboratories.

The piezo-drill generates a series of axial force pulses and the zona of the oocyte 508 is pierced by the tearing effect of the tip of the injection pipette 502. The tip is then cleared from the possible remnants of the zona and reinserted for the piercing of the membrane of the oocyte 508. The injection pipette 502 is pushed considerably into the oocyte 508, swaging the membrane and sufficiently stretching it. The piezo-drill is triggered once more with a lower amplitude piezo pulse, which pierces the membrane of the oocyte 508. Then the sperm is injected and the pipette is pulled out, completing the microinjection, and initiating the fertilization. As mentioned above, during the piercing process, piezo impulses can generate excessive lateral oscillations at the tip of the injection pipette 502. The amplitude of the lateral oscillations is reduced to a certain extent due to high inertia of mercury placed in the tip of the injection pipette 502.

The above-described procedure can increase the yield to 68% for mouse at blastocyst stage (Dozortsev 1998, Yanagida 1998). These rates display significant improvement over the conventional ICSI. On the other hand, the mercury is a highly toxic substance and its usage is extremely restricted. Despite this constraint, the procedure is adapted for most of the ICSI applications (Kawase 2002). In the meantime, efforts to substitute other heavy fluids for mercury have largely proved unsuccessful.

As described in the following references, establishing an effective ICSI technique without using mercury remains a very important requirement for transgenic laboratories around the world where ICSI is required to maintain valuable genetically-altered mutant mouse strains: Comp. Med., Vol. 55, pp. 140-144 (2005) by T. Kaneko and N. Nakagata (Kaneko 2005); PNAS, Vol. 98, pp. 13501-13506 (2001) by H. Kusukabe et al. (Kusukabe 2001); and Reproduction, Vol. 133, pp. 919-929 (2007) by M. W. Li et al. (Li 2007). For example, and as described in the following reference, techniques are desired to permit an operator to separate the sperm head from the tail before injection, since neither the sperm tail nor the centrosome in the neck region is required for ICSI-induced fertilization and embryo development: Biol. Reprod.; Vol. 55, pp. 789-795 (1996) by S. Kuretake et al. (Kuretake 1996). In addition, techniques are desired to permit the ICSI injection pipette to penetrate through the zona and break the oolemma easily. Still further, ICSI techniques are desired in which the ICSI procedure itself does not damage the sperm DNA or the oocyte spindle-chromosome complex.

### SUMMARY

Described herein in accordance with the present disclosure is a microinjection device comprising an injection element and a rotational motor. The injection element is rotatable about a longitudinal axis by the rotational motor, and is adapted to penetrate a target. In accordance with embodiments of the present disclosure, the injection element may include a beveled or spiked distal end adapted to permit the injection element to penetrate a target, and may be a micropipette, cannula, or needle. In accordance with embodiments of the present disclosure, the rotational motor may be adapted to rotate the injection element alternately clockwise and counterclockwise about the longitudinal axis, may be adapted to rotationally oscillate the injection element about the longitudinal axis, and may be a micromotor, such as a micromotor for rotating the injection element in alternate directions about the longitudinal axis through a range of between about 0.5 degrees and about 10 degrees peak-to-peak (e.g., a range of about 0.5 degrees and about 2 degrees peak-to-peak), and such as a micromotor for oscillating the injection element about the longitudinal axis with a frequency of about 10-500 cycles per second. In accordance with embodiments of the present disclosure, the microinjection device may further include an injection element holder that couples the injection element to the rotational motor, and may provide injectable materials to the injection element. In accordance with embodiments of the present disclosure, the microinjection device may further include means for manipulating the injection element, e.g., a micromanipulator, and the target may be selected from the group consisting of a cell, cell nucleus, embryo, ovum, oocyte, and zygote.

Also described herein in accordance with the present disclosure is a microinjection system comprising a microinjection device and a control unit. The control unit is for controlling a rotational amplitude and a frequency of oscillation of the injection element. The microinjection device includes an injection element and a rotational motor. The injection element is rotatable about a longitudinal axis by the rotational motor, and is adapted to penetrate a target. The microinjection system may further include an injection element positioner, e.g., a micromanipulator, for translatatably moving the injection element relative to the target. The microinjection system may further include means for manipulating the target during an injection procedure. In embodiments, such means for manipulating the target may include a holding pipette, and a micromanipulator coupled to the holding pipette, the micromanipulator being adapted to manipulate the holding pipette during an injection procedure for purposes of at least one of stabilizing the target, and moving the target.

Additionally, a method for penetrating a target to facilitate injecting material therein is provided in accordance with the present disclosure. The method includes providing the material to an injection element, contacting the target with a distal end of the injection element, rotating the injection element about a longitudinal axis (e.g., a longitudinal axis defined by the injection element) to form a hole in the target, and penetrating the target with the injection element via the hole formed in the target. In accordance with embodiments of the present disclosure, the rotating step may include rotating the injection element alternately clockwise and counterclockwise about the longitudinal axis in an oscillatory manner to form the hole in the target, e.g., by causing the injection element to oscillate within a range of angular motion of between about 0.5 degrees and 10 degrees peak-to-peak (e.g., between about 0.5 degrees and about 2 degrees peak-to-peak), and may further include the step of expelling the material into the penetrated target. In accordance with embodiments of the present disclosure, the step of contacting the target with a distal end of the injection element may include one or both of translationally moving the injection element toward the target and translationally moving the target toward the injection element

Additionally, a method for performing intra-cytoplasmic sperm injection (ICSI) is provided in accordance with the present disclosure. The ICSI procedure includes providing a solution comprising sperm to an injection element, contacting an oocyte with a distal end of the injection element, rotating the injection element alternately clockwise and counterclockwise about a longitudinal axis to form a hole in the oocyte, penetrating the oocyte with the distal end of the injection element via the hole formed in the oocyte, and expelling the solution comprising sperm into the penetrated oocyte. In accordance with embodiments of the present disclosure, the step of rotating the injection element may include causing the injection element to oscillate within a range of angular motion of between about 0.5 degrees and about 10 degrees peak-to-peak (e.g., between about 0.5 degrees and about 2 degrees peak-to-peak), and the step of contacting an oocyte with a distal end of the injection element may include deflecting inward a cell membrane of the oocyte.

Additional advantageous features, functions and benefits of the disclosed apparatus, system and treatment methods will be apparent from the detailed description which follows, particularly when read in conjunction with the appended figures.

### BRIEF DESCRIPTION OF THE FIGURES

To assist those of ordinary skill in the art in making and using the disclosed apparatus/systems, reference is made to the accompanying figures, wherein:

FIG. 1 is a representation of components involved in a standard cellular injection procedure;

FIG. 2 is a schematic representation of a cellular injection procedure commonly employed during in-vitro fertilization in which the associated micropipette is oscillated along a longitudinal direction;

FIG. 3 is a representation of time-dependant data of a micropipette tip arising from a longitudinally-directed oscillation of the micropipette in air medium;

FIG. 4 is a representation of time-dependant data of a micropipette tip arising from a longitudinally-directed oscillation of the micropipette in cell medium;

FIG. 5 is a schematic, side elevation depiction of a known cellular injection procedure including piezo-drill actuation of the associated injection pipette;

FIG. 6 is a schematic representation of an injection procedure in accordance with embodiments of the present disclosure;

FIG. 7 is a schematic representation of a microinjection device in accordance with embodiments of the present disclosure;

FIG. 8 is a schematic representation of another microinjection device in accordance with embodiments of the present disclosure;

FIG. 9 is a schematic representation of a microinjection system in accordance with embodiments of the present disclosure;

FIG. 10 is a schematic representation of another microinjection system in accordance with embodiments of the present disclosure;

FIG. 11 is a flow chart illustrating an exemplary control logic between a user interface and a motor control of the microinjection system of FIG. 10;

FIG. 12 is a graphical depiction of reference and actual rotational oscillatory trajectories associated with an exemplary cell-piercing protocol for use in conjunction with the microinjection system of FIG. 9 in accordance with embodiments of the present disclosure;

FIG. 13 is a detail of the FIG. 11 graphical depiction of oscillatory trajectories;

FIG. 14 is a flow chart illustrating a method of microinjection in accordance with embodiments of the present disclosure;

FIGS. 15, 16, 17, and 18 collectively present a series of photographs of respective pre-penetration, penetration, penetrated, and post-penetration stages of a cellular injection procedure in accordance with embodiments of the present disclosure;

FIG. 19 presents a series of photographs of an intracytoplasmic sperm injection procedure (ICSI) in accordance with the present disclosure and performed using the microinjection system of FIG. 10;

FIG. 20 presents a photograph of blastocysts derived from an ICSI procedure in accordance with embodiments the present disclosure and performed using the microinjection system of FIG. 10.

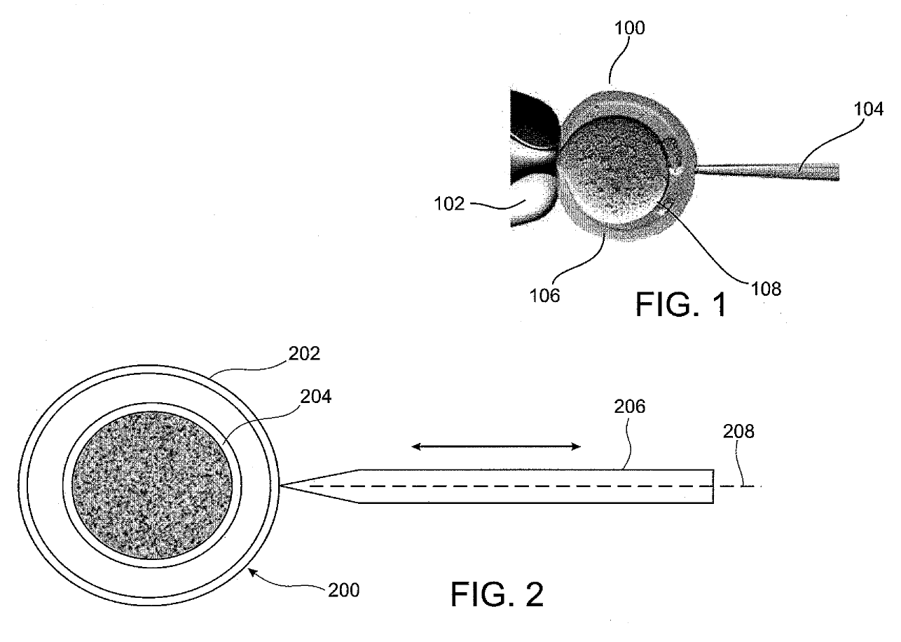

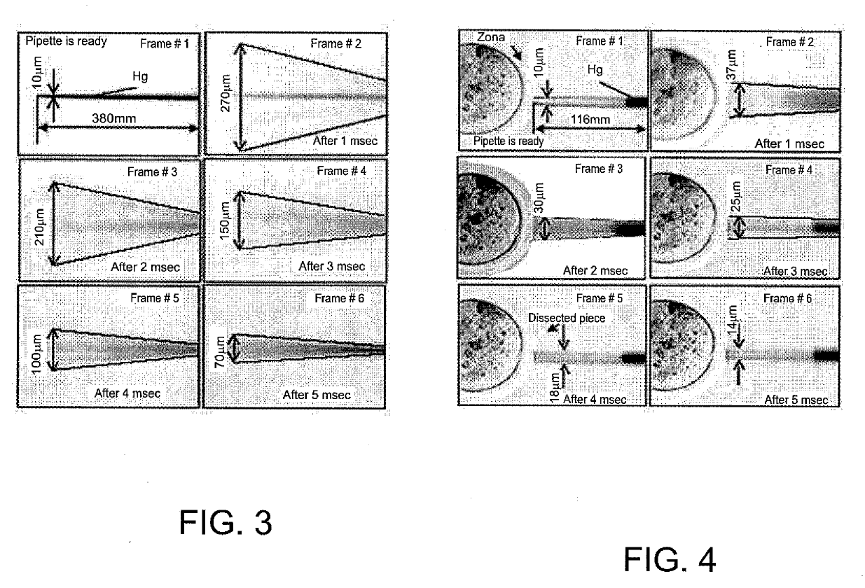

### DESCRIPTION

The present disclosure provides microinjection devices and methods that facilitate target penetration without the need to drive the micropipette into longitudinally-directed oscillations. In accordance with embodiments of the present disclosure, because such longitudinally-directed oscillations are not needed, the micropipette tip need not necessarily experience destructive transverse motion when interfacing with the cell.

In accordance with embodiments of the present disclosure, a procedure is provided in which the injection element oscillates in a rotational manner, e.g., alternatively rotating in opposite rotational directions as the injection element encounters the injection target.

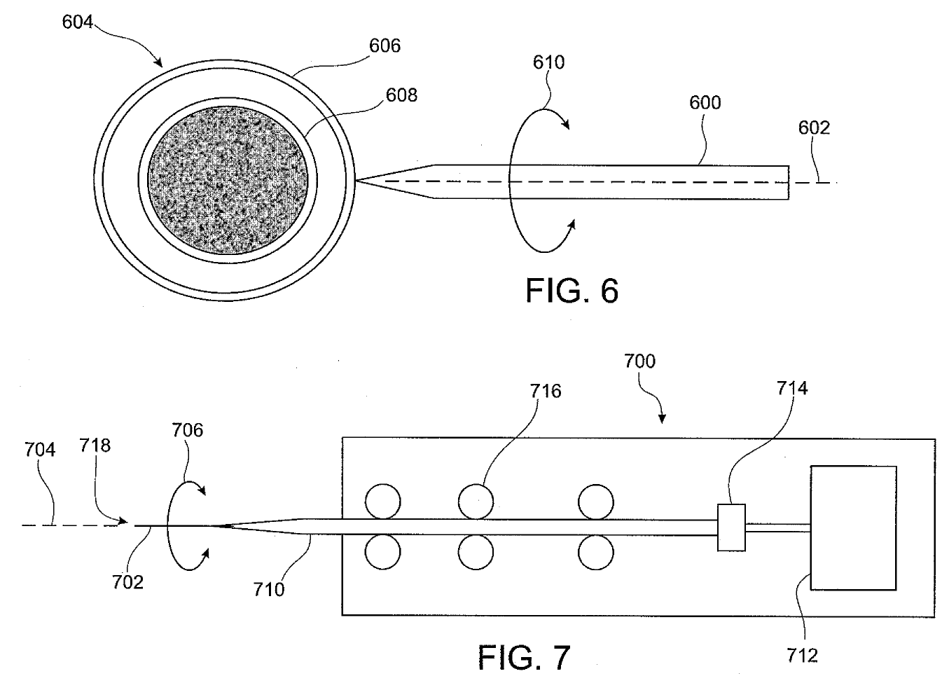

Referring now to FIG. 6, an injection element 600 may include or define a longitudinal axis 602, and may be caused to interface with a target. As shown in FIG. 6, the target may be a cell 604, which cell 604 may include a zona pelucida 606, and a cell membrane 608 or oolemma. In accordance with embodiments of the present disclosure, and as generally indicated via reference numeral 610 in FIG. 6, the injection element 600 may be rotationally oscillated about the longitudinal axis 602 during a related process of injecting matter into the cell 604. For example, the present applicants have observed wherein such rotational oscillation of the injection element 600 can be effective to facilitate penetration by the injection element 600 of the zona pelucida 606 and the cell membrane 608 of the cell 604, while also beneficially reducing and/or limiting an associated structural damage thereto to an extent sufficient to improve cell viability and reduce associated failure rates.

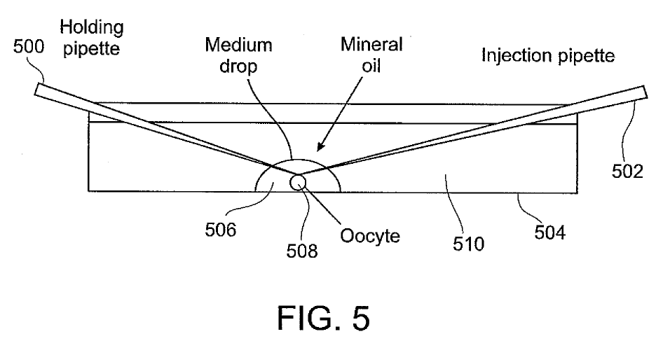

Turning now to FIG. 7, a device 700 for performing microinjection processes or procedures, e.g., such as described herein with respect to FIG. 6, is provided in accordance with embodiments of the present disclosure. The device 700 may include an injection element 702, wherein the injection element 702 may include or define or be associated with a longitudinal axis 704, and may be caused to interface with a target (not shown). As generally indicated via reference numeral 706 in FIG. 5, the injection element 702 may be rotationally oscillated about the longitudinal axis 704 during a related injection process. The device 700 may further include: i) a frame or body 708, ii) a holder 710 for receiving and securely holding the injection element 702 and rotationally mounted with respect to the body 708, and iii) a motor 712 mounted with respect to the body 708 and operably coupled to the holder 710 for rotating the holder 710 (e.g., relative to the body 708) and/or for rotating the injection element 702 about the longitudinal axis 704, thereby facilitating penetration by the injection element 702 of a target (not shown) in accordance with embodiments of the present disclosure. The device 700 may further include: iv) a force transducer 714 for operably coupling the motor 712 to the holder 710 and/or to the injection element 702 (e.g., to facilitate the above-described rotation thereof); and v) a positioner 716 operably coupled to the holder 710 for facilitating precise positioning of the holder 710, and/or of the injection element 702 (e.g., longitudinally relative to the body 708, and/or transversely relative thereto). As shown in FIG. 6, the holder 710 may couple the injection element 702 to the motor 712 via the force transducer 714. In accordance with embodiments of the present disclosure, the longitudinal axis 704 about which the injection element 702 is adapted to be rotated may be defined by any one or more of the injection element 702, the body 708, the holder 710, the motor 712, the force transducer 714, and the positioner 716.

The injection element 702 may include a tip 718 adapted to engage and penetrate a target. In accordance with embodiments of the present disclosure, the injection element 702 may be a micropipette, a cannula, a needle, another similar component, or a combination of one or more such components. For example, the injection element 702 may be implemented by one or more microinjection elements, such as a glass rod or a glass capillary tube heated and drawn to a microscopic point in a vicinity of the tip 718. Glass may be a desirable material for the injection element 702 due to the characteristics of the material being chemically inert, ductile, and/or sterilizable. For example, and as will be appreciated by those of ordinary skill in the art, the injection element 702 may be fabricated from a type of glass that will allow the injection element 702 to be drawn to a submicron point at the tip 718. In accordance with embodiments of the present disclosure, microneedles and/or micropipettes may be manually manufactured in a lab. The anticipated targets may include, for example, cells, cell nuclei, embryos, ova, oocytes, and zygotes.

In accordance with embodiments of the present disclosure, the injection element 702 may include a beveled point at its target penetrating tip 718. More particularly, the present applicants have observed wherein a beveled point at the tip 718 of the injection element 702 may be advantageous for limiting structural damage to the target (e.g., to cell structure) arising during target penetration by the injection element 702. Other shapes for the tip 718 are possible, such as a jagged-edged shape, and/or a non-beveled shape.

The injection element 702 may be alternatively rotatable about the longitudinal axis 704 in respective opposite (e.g., clockwise and counter-clockwise) directions by the motor 712. Specifically, the motor 712 may be operably coupled to the injection element via the force transducer 714 and/or the holder 710 so as to permit the motor 712 to rotationally oscillate the injection element about the longitudinal axis 704, such that the injection element 702 selectably oscillates rapidly between rotational motion thereabout in a first rotational direction, and rotational motion thereabout in a second (e.g., opposite) rotational direction.

The motion of the injection element 702 may be dictated by a combination of oscillation amplitude and oscillation frequency. In accordance with embodiments of the present disclosure, the amplitude of the oscillations may be a fraction of a degree, or equal to or greater than a whole degree, and/or up to 10 degrees. Other oscillation amplitudes are possible. Undesired transverse motion at the injection element tip may be minimized in accordance with embodiments of the present disclosure by providing relatively small oscillation amplitudes, such as 1 degree or less, with respect to each direction of rotation about the longitudinal axis 704.

The frequency of the oscillations of the injection element 702 about the longitudinal axis 704 may be restricted by machine limitations. For example, the frequency of the oscillations may be limited to a maximum of about 100 Hz in circumstances in which instrumentation limitations exist with respect to providing alternating rotations at frequencies higher than about 100 Hz. The amplitude and frequency of the oscillations of the injection element 702 about the longitudinal axis 704 may further vary depending on the particular characteristics of the target material.

Lower frequencies, or slower rotations, than about 100 Hz may be provided with respect to the oscillations of the injection element 702 about the longitudinal axis 704. In at least some circumstances, one or more such lower frequencies may allow penetration of certain target surfaces. It may be noted, however, that certain target surface adhesion forces may tend to interact more strongly with the injection element 702 in the presence of a relatively lower oscillating frequency, potentially contributing to greater damage to the target surface. By rotationally oscillating at an appropriately high frequency, the injection element 702 should, however, pierce the target smoothly, and/or causes an acceptably limited degree of damage thereto.

The holder 710 may position or stabilize the injection element 702, and may also hold and supply the materials to be injected into the target. The holder 710 can also contain components that deliver materials to the injection element 702, and thus may assist in carrying out the injection.

The holder 710, and/or the injection element 702 itself, may be positioned or stabilized, e.g., with respect to the body 708, by the positioner 716. In accordance with embodiments of the present disclosure, the positioner 716 may be a micromanipulator. For example, the positioner 716 may be a micromanipulator serving to scale an operator's motion from about 100:1 to 10000:1, allowing an operator to position or stabilize the injection element on a microscopic level through a micromanipulator control unit. Such a device for positioning or stabilizing the injection element 702 may permit an individual to execute microscopic movements, e.g., with respect to the body 708 or otherwise, in a controlled and steady manner. Examples of these manipulative devices may include hydraulic instruments, electromagnetic instruments, and/or any combination thereof. Other types of micromanipulators are possible. In accordance with embodiments of the present disclosure, the positioner 716 may be capable of providing motion control with respect to the injection element 702 and/or the holder 710 in the range of a few millimeters in any direction, and/or may be capable of positional accuracy to within a few microns, to within less than a tenth of a micron, and/or to within one hundredth of a micron.

In accordance with embodiments of the present disclosure, the motor 712 may be a micromotor adapted to rotate the injection element 702 in alternative directions. For example, such a micromotor may be adapted to alternatively rotate the injection element 702 about the longitudinal axis 704 through an oscillation amplitude amounting to no greater than a fraction of a degree. For another example, such a micromotor may be adapted to alternatively so rotate the injection element through an oscillation amplitude of up to 10 degrees. Appropriate micromotors for use in the device 700 may include, for example, a precision DC servo motor with a capacity to rotate the injection element 702 in an oscillatory fashion with a frequency of above 100 Hz with corresponding amplitudes of less than 2 degrees. Appropriate micromotors for use in the device 700 may provide resolution within a fraction of a degree, e.g., providing a resolution of less than one half (0.5) of a degree. In accordance with embodiments of the present disclosure, the oscillations provided by such an appropriate micromotor may occur in repetitive intermittent periods lasting anywhere from a fraction of a second to several seconds, to a continuous period of oscillations.

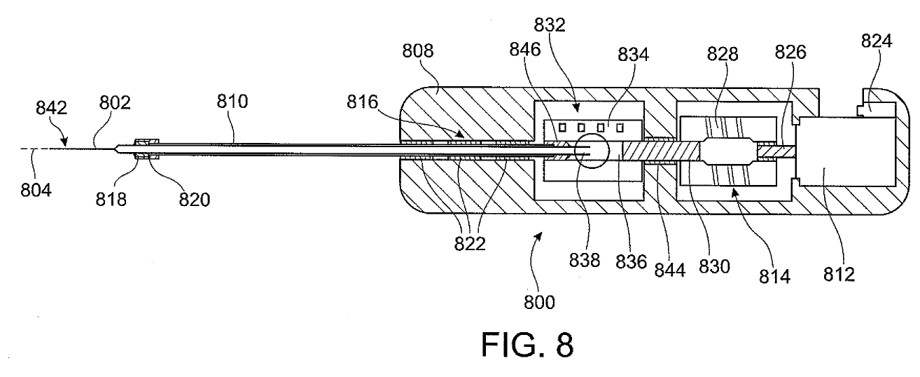

Turning now to FIG. 8, a device 800 for performing microinjection processes or procedures is provided in accordance with embodiments of the present disclosure. The device 800 may be an implementation of the device 700 described above with reference to FIG. 7. More particularly, the device 800 and the device 700 may include similar and/or common features, functions, structures, and/or components, at least including wherein the device 800 may comprise an injection element 802 including or defining or being associated with a longitudinal axis 804 about which the injection element 802 may be rotationally oscillated during a related injection process, a frame or body 808, a holder 810 for receiving and securely holding the injection element 802 and rotationally mounted with respect to the body 808, a motor 812 mounted with respect to the body 808 and operably coupled to the holder 810 for rotating the holder 810 and/or the injection element 802, a force transducer 814 for operably coupling the motor 812 to the holder 810 and/or to the injection element 802, and a support 816 operably coupled to the holder 810 for precisely movably mounting the holder 810 to the body 808. In accordance with embodiments of the present disclosure, the longitudinal axis 804 about which the injection element 802 is adapted to be rotated may be defined by any one or more of the injection element 802, the body 808, the holder 810, the motor 812, the force transducer 814, and the support 816.

Still referring to FIG. 8, the injection element 802 may be a glass pipette, the holder 810 may be a pipette holder, and the injection element may be attached to the holder 810 via a tip screw 818 and an inner seal 820 with respect to which the tip screw 818 is adapted to be mounted. The positioner 816 may include one or more bushings 822 mounted with respect to the housing 808 and adapted to support and/or position the holder 810 within the housing 808, and/or to guide the holder 810 (e.g., with respect to rotational motion with respect to the housing 808).

The motor 812 may be a micromotor, and may include an electrical port 824 to facilitate supplying power to the motor 812, and a shaft 826. The force transducer 814 may include a coupling 828, which may be dimensionally flexible (e.g., deflectable in twist), and a shaft 830, wherein the shaft 830 may be substantially rigid.

The device 800 may further include a junction 832 mounted with respect to the housing 808 between the force transducer 814 and the holder 810. The junction 832 may include a coupling 834 fastened to the shaft 830 of the force transducer, wherein the coupling 834 may define a cavity 836, and may be substantially rigid to facilitate the transmission of torquing forces from the force transducer 814 to the holder 810 across the junction 832. The device 800 may further include a tube 838 extending within the cavity 836 and longitudinally through the holder 810, and terminating at a proximal end 840 of the injection element 802. The tube 838 may be adapted to supply a material or materials to the holder 810 and/or to the injection element 802 for facilitating the ejection of a material or materials from a distal end 842 of the injection element 802 (e.g., during a related injection or microinjection procedure).

The device 800 may further be supplied with a bushing 844 for rotationally mounting the shaft 830 of the force transducer 814 to the housing 808, and a tall screw 846 for mounting the holder 810 with respect to the coupling 834 of the junction 832, and/or for receiving the tube 838 from the coupling 834 and permitting the tube 838 to extend outward therefrom and into the holder 810.

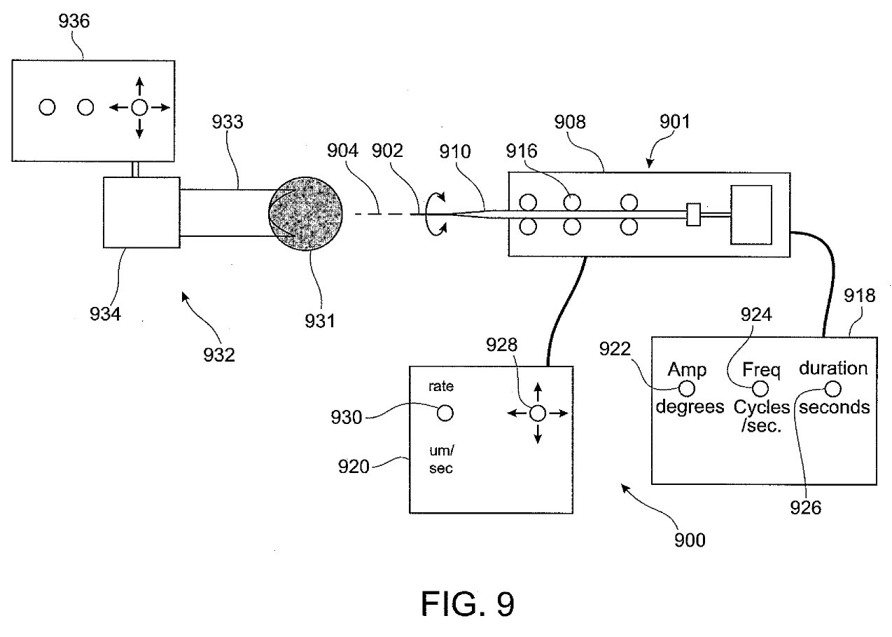

Referring now to FIG. 9, a system 900 is provided for use in conjunction with microinjection methods and procedures in accordance with embodiments of the present disclosure. The system 900 includes a device 901 for performing microinjection processes or procedures, e.g., such as described herein with respect to FIG. 6, in accordance with embodiments of the present disclosure. The device 901 may be an implementation of the device 700 described above with reference to FIG. 7. More particularly, the device 901 and the device 700 may include similar and/or common features, functions, structures, and/or components, at least including wherein the device 901 may comprise an injection element 902 including or defining or associated with a longitudinal axis 904 about which the injection element 902 may be rotationally oscillated during a related injection process, a frame or body 908, a holder 910 for receiving and securely holding the injection element 902 and rotationally mounted with respect to the body 908, a motor 912 mounted with respect to the body 908 and operably coupled to the holder 910 for rotating the holder 910 and/or the injection element 902, a force transducer 914 for operably coupling the motor 912 to the holder 910 and/or to the injection element 902, and a positioner 916 operably coupled to the holder 910 for facilitating precise positioning of the holder 910, and/or of the injection element 902. In accordance with embodiments of the present disclosure, the longitudinal axis 904 about which the injection element 902 is adapted to be rotated may be defined by any one or more of the injection element 902, the body 908, the holder 910, the motor 912, the force transducer 914, and the positioner 916.

Still referring to FIG. 9, the system 900 further includes a control unit 918 for permitting a user to control the motor 912 of the device 901, and a control unit 920 for permitting a user to control the positioner 916 of the device 901. The control unit 918 may include a controller 922 and a controller 924 for respectively permitting the user to selectively adjust an amplitude and a frequency of the rotational oscillations of the injection element 902 about the longitudinal axis 904. Amplitude refers to how much or how far the injection element will rotate in each direction. In accordance with embodiments of the present disclosure, symmetric oscillations in each direction with an amplitude of between less than one degree to 10 degrees are appropriate for many microinjection applications. In embodiments of the system 900 in which the motor 912 is a micromotor, amplitude may be restricted by the resolution of the particular micromotor used, consistent with the oscillations being uniform in each direction. In this regard, a sufficiently large amplitude may tend to create undesired transverse motion at the tip of the injection element 902. In accordance with embodiments of the present disclosure, amplitudes of between about 0.5 degrees and about 10 degrees and frequencies of between about 10 Hz to about 200 Hz are feasible for most biological applications, with amplitudes of between about 0.5 degrees and about 5 degrees and a frequency of about 100 Hz being appropriate in many applications (e.g., for penetrating a cell with a glass micropipette).

The control unit 918 may further include a controller 926 for controlling a duration (e.g., a length of time) of oscillatory rotation of the injection element 902 about the longitudinal axis 904, a duration of a dwell period between successive instances of such oscillatory motion, or both. In accordance with embodiments of the present disclosure, each period of oscillation may be of between about 0.5 second and about 10 seconds in duration.

The control unit 920 may include respective controllers 928 and 930 for operating or controlling the positioner 916 of the device 901. The controller 928 may be operable to control a direction of translational motion of the injection element 902, e.g., with respect to the body 908, and/or with respect to a target 931, wherein the latter may be a mouse oocyte. The controller 930 may be operable to control a speed of such translational motion.

In accordance with embodiments of the present disclosure, the system 900 further includes means for manipulating the target 931 during an injection procedure. Such means can include, e.g., any appropriate means for positioning and/or stabilizing the target 931. For instances, the system 900 may include a target manipulation device 932, wherein the target manipulation device 932 may include a holding pipette 933, and a positioner 934 coupled to the holding pipette 933 and adapted to manipulate the holding pipette 933 for purposes of stabilizing, moving, and/or otherwise manipulating the target 931. As shown in FIG. 9, the system 900 may further include a control unit 936 coupled to the positioner 934 for controlling a movement or other behavior of the positioner 934 and/or for controlling the target manipulation device 932 generally. In accordance with embodiments of the present disclosure, the positioner 934 may be or include a micromanipulator. In accordance with embodiments of the present disclosure, the positioner 934 may include one or more Eppendorf manipulators, and/or one or more Narishiga manipulators. The use of other and/or different manipulators to embody the positioner 934 is possible.

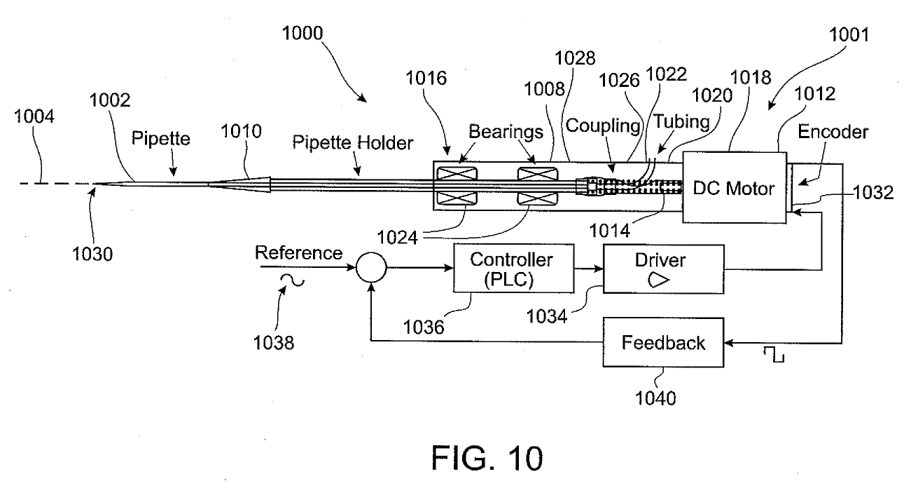

Referring now to FIG. 10, a system 1000 is provided for use in conjunction with microinjection methods and procedures in accordance with embodiments of the present disclosure. The system 1000 includes a device 1001 for performing microinjection processes or procedures, e.g., such as described herein with respect to FIG. 7, in accordance with embodiments of the present disclosure. The device 1001 may be an implementation of the device 700 described above with reference to FIG. 7. More particularly, the device 1001 and the device 700 may include similar and/or common features, functions, structures, and/or components, at least including wherein the device 1001 may comprise an injection element 1002 including or defining or associated with a longitudinal axis 1004 about which the injection element 1002 may be rotationally oscillated during a related injection process, a frame or body 1008, a holder 1010 for receiving and securely holding the injection element 1002 and rotationally mounted with respect to the body 1008, a motor 1012 mounted with respect to the body 1008 and operably coupled to the holder 1010 for rotating the holder 1010 and/or the injection element 1002, a force transducer 1014 for operably coupling the motor 1012 to the holder 1010 and/or to the injection element 1002, and a support 1016 operably coupled to the holder 1010 for precisely movably mounting the holder 1010 to the body 1008. In accordance with embodiments of the present disclosure, the longitudinal axis 1004 about which the injection element 1002 is adapted to be rotated may be defined by any one or more of the injection element 1002, the body 1008, the holder 1010, the motor 1012, the force transducer 1014, and the support 1016.

Referring still to FIG. 10, the motor 1012 may be a micromotor, such as a precision DC-servo motor, and may include a shaft 1018. The force transducer 1014 may include a coupling 1020, which may be dimensionally flexible (e.g., deflectable in twist and/or bendable) in response to the application thereto of torquing forces by the motor 1012 via the shaft 1018, and/or in response to such inertial and/or frictional forces as may arise elsewhere in the device 1001 and as may tend to resist or at least partially oppose the transmission of torquing forces to the holder 1010. The coupling 1020 may further facilitate efficient operation of the device 1001 in circumstances in which the shaft 1018 of the motor 1012 and the holder 1010 are to at least some extent axially misaligned. The coupling 1020 may further define a cavity 1022. The support 1016 may include a set of bearings 1024 within which the holder 1010 may be embedded within the body 1008.

The device 1001 may further include a tube 1026 extending within the cavity 1022 and terminating at a proximal end 1028 of the holder 1010. The tube 1026 may be adapted to supply a material or materials to the holder 1010 and/or to the injection element 1002 for facilitating the ejection of a material or materials from a distal end 1030 of the injection element 1002 (e.g., during a related injection or microinjection procedure).

The system 1000 may further include an encoder 1032, which may be attached to the shaft 1018 of the motor 1012, a driver 1034 for driving the motor 1012, and a controller 1036. A reference signal 1038 may be harmonic A sin(2πft), where A (deg) is the amplitude of the oscillations and f(Hz) is the frequency of the oscillations. A pure harmonic reference trajectory may be purposely selected to avoid an unnecessary excitation of the natural vibration modes of the injection element 1002 (e.g., in cases in which the injection element 1002 is a drawn pipette). Such a trajectory is relatively easy to generate and to implement. The encoder 1032, which may be an incremental encoder, may be capable of generating a feedback signal 1040 for providing positional feedback. The driver 1034 may be a linear amplifier. The device 1001 may be activated via a start button (not shown), e.g., a foot switch.

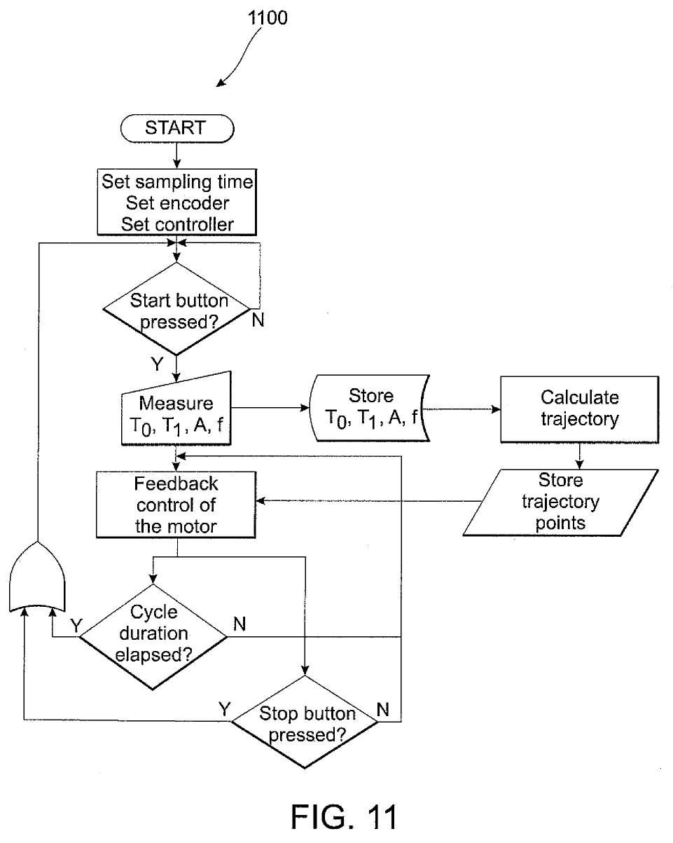

A PID (proposition-integral-derivative) class of control logic may be used in accordance with the present disclosure for making the motor 1012 track the harmonic reference trajectory. For example, the encoder 1032 may generate 512 pulses/revolution when used in quadrature mode, such that it determines the sensitivity of position feedback with 0.175° increments. A flow chart 1100 shown in FIG. 11 depicts an exemplary control logic between the user interface and the motor control. A program for implementing the steps of the flow chart 1100 may also set the sampling rate for the motor control, as well as PID control gains, and encoder settings. After the first cycle, the system 1000 may be ready for user inputs, which are elaborated below.

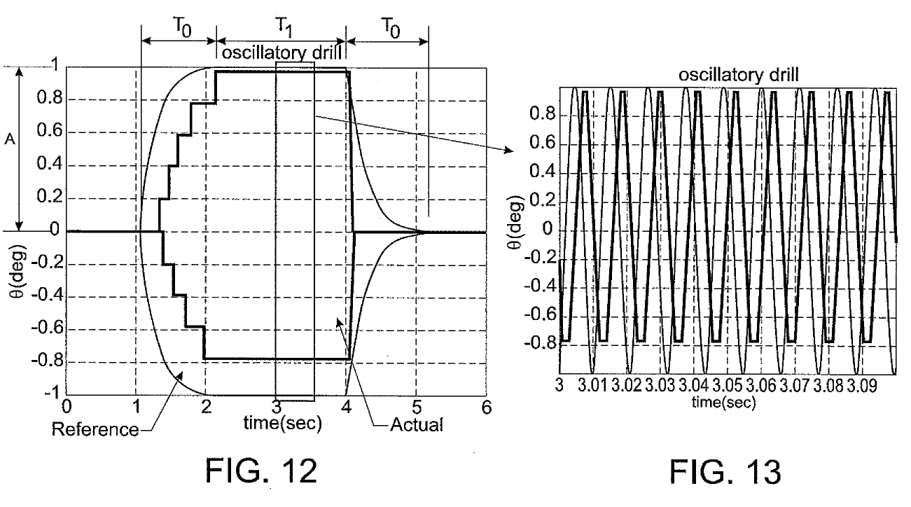

The device 1001 may be associated with a new cell-piercing protocol. In order to avoid the array of natural frequencies, a soft start of the motor 1012 may be deployed by smoothly increasing the amplitude of the oscillatory reference signal 1038 with a fixed frequency instead of maintaining a fixed amplitude harmonic sweep. The parameters of the device 1001 may be the amplitude of the rotational oscillation (A, deg), frequency (f, Hz), rising time (T₀, s) and duration (T₁, s). When the foot-switch is pressed, the parameters of this variable-amplitude harmonic reference trajectory may be communicated from manually selected potentiometer inputs on the controller 1036. The trajectory for the injection element motion may be generated numerically and stored. The amplitude of the oscillation, with frequency f may increase from 0 to desired A in T₀ seconds following a smooth first-order curve rise. The injection element 1002 may oscillate for T₁ seconds at that amplitude and the amplitude may decreases from A to 0 in T₀ seconds following the fixed amplitude phase, as shown in FIGS. 12 and 13. This protocol avoids undesirable jolts at the pipette-cell interface. In the case of successful piercing before the completion of the desired trajectory sequence, the operator may manually interfere via a stop button (not shown). The control structure may be configured to assure that the absolute angular position of the injection element 1002 returns to zero at the start of each cycle in order to prevent wrap-around of the tube 1026.

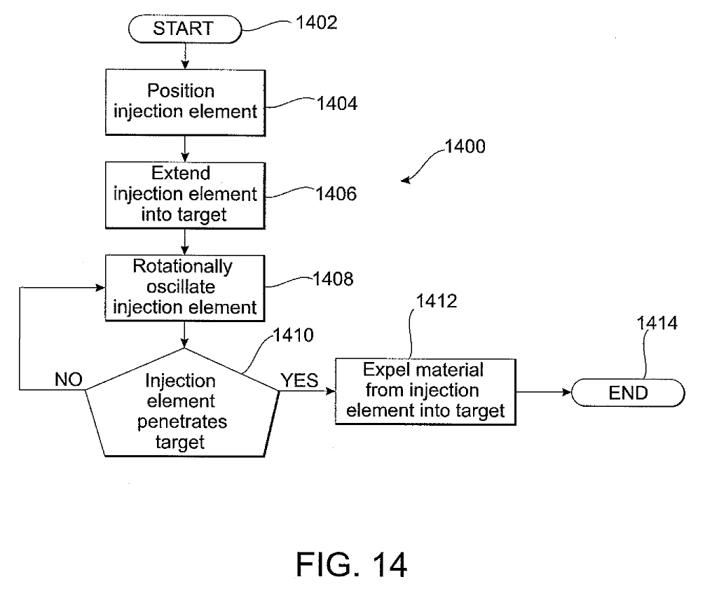

Referring now to FIG. 14, a method is provided for injecting material into a target in accordance with embodiments of the present disclosure. More particularly, a flow chart 1400 describing such a method is depicted in FIG. 14, wherein the steps of the method may be performed using the system 900 of FIG. 9. The method 1400 begins with a step 1402, and proceeds to a step 1404. Referring to FIGS. 9 and 14, the step 1404 may include providing the material to the injection element 902 of the device 901 and positioning the injection element 902 in proximity to the target 931, such as a mouse oocyte. More particularly, the injection element 902 may be advanced toward the target 931 a sufficient distance such that a the distal tip of the injection element 902 touches the target 931.

Proceeding now to a step 1406, the injection element 902 may be extended further toward the target 931, e.g., such that the distal tip of the injection element 902 forms a dimple in the cell membrane of the target 931. More particularly, an operator may operate the control unit 920 and/or the controllers 928, 930 thereof to move the injection element 902 toward the target 931 to an extent of a predetermined distance, and/or at a predetermined speed, wherein each of the predetermined distance and speed may be calculated to preserve the integrity of the cell membrane pending a further injection step.

Proceeding now to a step 1408, the injection element 902 may be rotated about the longitudinal axis 904, creating a relative displacement as between the injection element 902 and the target 931, and achieving the piercing of the target 931. For example, an operator may operate and/or control the motor 912 to cause the motor 912 to rotationally oscillate the injection element 902 about the longitudinal axis 904, causing a sharp edge at the distal tip of the injection element 902 in contact with the target 931 to abrade the zona pellucida and/or through the cell membrane of the target 931 for purposes of boring or otherwise forming a hole therein via which the injection element 902 may penetrate the target 931. The motor 912 may be operated such that the rotational oscillation of the injection element 902 ceases after a predetermined time period and/or after a predetermined number of sessions of such rotational oscillations divided by respective pauses in such motion. The oscillations can occur, for example, in isolated bursts lasting between 0.5 and 10 seconds. Multiple iterations of these oscillation episodes can be used to achieve the desired result of penetration of the target 931. The method 1400 can include iteratively and/or continuously rotating the injection element 902 counterclockwise and/or clockwise about the longitudinal axis 904, e.g., in an oscillatory manner, without stopping until the target 931 is penetrated and/or until the operator decides to cease such rotation.

In a next step 1410, a determination is made whether the injection element 902 has successfully penetrated the target 931 as a result of the rotational oscillation or ‘drilling’ step 1408. If not, the method 1400 may proceed back to the step 1406, commencing further rotational oscillation or drilling. Alternatively, the method 1400 may proceed back to the step 1404, to commence further advancement of the injection element 902 toward the target 931 prior to a further session of rotational oscillation or drilling. If so, the method 1400 may proceed to a step 1412, in which material, e.g., a partial or complete sperm, is expelled from a distal tip of the injection element 902 into the target 931. The method 1400 may then proceed to a step 1414, at which step the method 1400 ends.

Once example of the method 1400 involves intra-cytoplasmic sperm injection (ICSI), in which a sperm is injected into an appropriate cell, such as an oocyte, to fertilize it. A solution comprising sperm may be provided to the injection element 902, and the injection element 902 is positioned in the proximity of an exterior of an oocyte, which is stabilized by a holding pipette. The injection element 902 oscillates counterclockwise and clockwise around its longitudinal axis 904. Intermittent oscillatory rotations continue as the injection element 902 is extended into the oocyte and penetrates it. Once the injection pipette has penetrated the oocyte, the solution comprising sperm is expelled into the penetrated oocyte.

### EXAMPLES

### Example 1

### Penetration of Bovine Oocyte

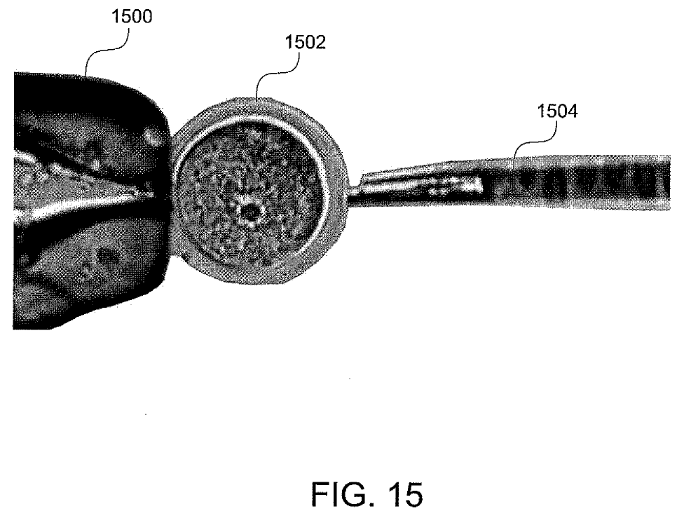

FIGS. 15, 16, 17 and 18 highlight four stages of an injection process using an injection apparatus as described herein. In this procedure, a glass micropipette was used with a micromotor that alternately rotated 1 degree clockwise and counterclockwise, with a frequency of 100 Hz. The rotational resolution of the micromotor was 0.17 degrees.

FIG. 15 shows the prepenetration stage. A holding pipette 1500 holds a bovine oocyte 1502 in position while an injection pipette 1504 is positioned at the surface of the oocyte outer membrane.

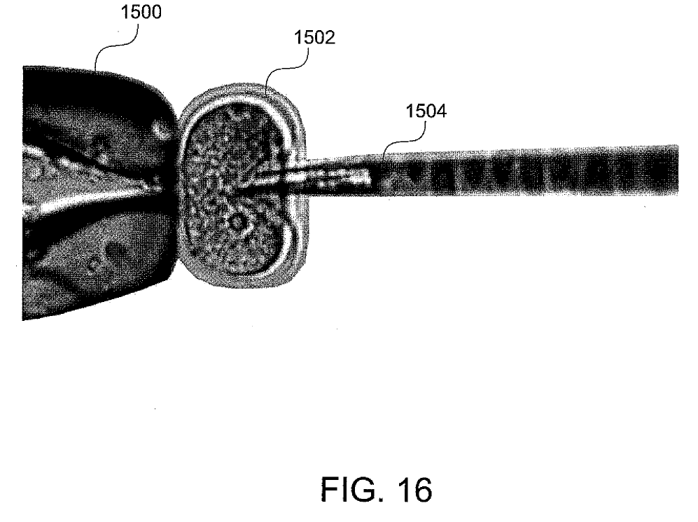

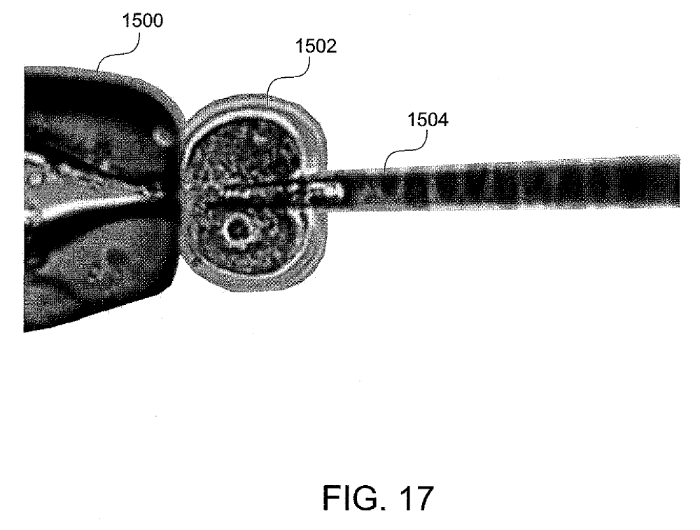

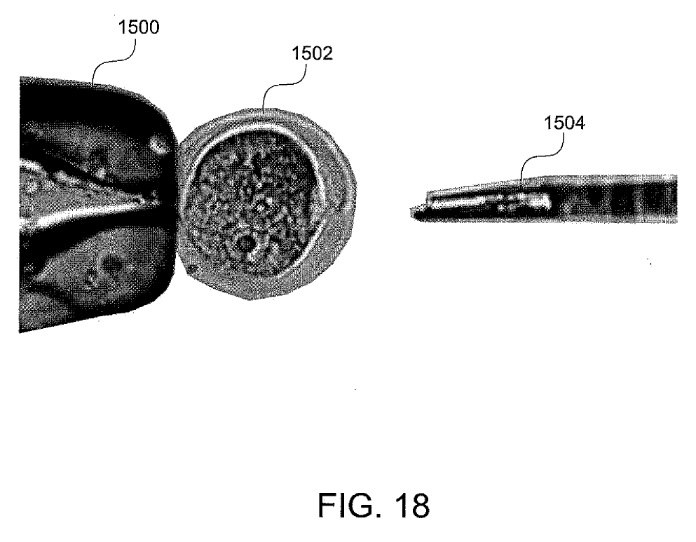

The penetration stage is shown in FIG. 16, where the injection pipette 1504 oscillates relative to the oocyte 1502 in order to form a hole in the oocyte 1502 (e.g., via a drilling action). FIG. 17 shows the penetrated stage where the injection pipette 1504 has fully penetrated the oocyte 1502. This is the stage where the material held in the injection pipette 1504 for injection is expelled into the oocyte 1502. Finally, FIG. 18 shows the post-penetration stage, after the injection pipette 1504 has been removed from the oocyte 1502.

### Example 2

### Penetration of Mouse Oocytes

A prototype was built in accordance with the example of the system 1000 shown and described herein with respect to FIGS. 10-13 and was used for performing ICSI on mouse oocytes of hybrid BDF1 strain at the Center for Regenerative Biology and the Department of Animal Science, University of Connecticut. The operational parameters of the system 1000, also referred to herein as Ros-drill©, were set at A=0.6°, f=100 Hz, T0=0.5 seconds, and T1=1 second for these preliminary biological experiments.

Before the ICSI tests, several different pipette styles were tried out, with the consideration being that the geometry of the injection element 1002 being capable of playing a crucial role in the exercise. Three different pipette varieties were compared while their internal and external diameters were kept fixed (5 μm and 7 μm, respectively).

A first pipette variety tried was that of Piezo-ICSI flat tip pipettes. Piercing was unsuccessful with this pipette due to flatness of the pipette tip.

A second pipette variety tried was that of jagged edged pipettes. Utilizing these pipettes, successful and repeatable penetration was obtained, but with poor or unsatisfactory healing of the damage on the membrane.

A third pipette variety tried was that of beveled and spiked human-ICSI pipettes. Such pipettes resulted in repeated piercing with minimal damage. All these tests were conducted by the first named applicant herein, who as an operator is from Mechanical/Electrical Engineering background and with no prior training in ICSI. An initial phase of a few weeks of introductory exposure (i.e., that was how long his ICSI training was) was sufficient for him to conduct the test listed in table 1 below.

TABLE 1
Set of recent experimental results on mouse BDF1

| | | | | | | |
|---|---|---|---|---|---|---|
| | # of | | Survival | | | Cleavage |
| Experiments | Oocytes | Survival | % | PN | Cleaved | % |

April 2007

| | | | | | | |
|---|---|---|---|---|---|---|
| 6-Apr | 60 | 25 | 42 | 25 | 22 | 88 |
| 10-Apr | 20 | 6 | 30 | 4 | 4 | 67 |
| 24-Apr | 44 | 13 | 30 | 9 | 9 | 69 |

May 2007

| | | | | | | |
|---|---|---|---|---|---|---|
| 3-May | 70 | 19 | 27 | 15 | 15 | 79 |
| 18-May | 96 | 31 | 32 | 25 | 22 | 71 |
| 22-May | 50 | 14 | 28 | 13 | 11 | 79 |
| 23-May | 72 | 20 | 28 | 19 | 17 | 85 |

During rotational oscillation of the pipette holder, the pipette tip was observed to have some whirling motion (e.g., in the air) and some ‘snaking’ motion when in contact with the target oocytes. However, these whirling and/or snaking effects generate limited (and acceptably small) lateral displacements depending on the eccentricity level. In order to avoid lateral displacements, the present applicants oscillated the pipette with very small angular amplitudes (e.g., 1° peak-to-peak) and at frequencies that are above natural frequencies of mode 1 and mode 2 (e.g., 90-100 Hz) and much lower than mode 3. Furthermore, due to the edge heights of the Petri dish the injection pipettes are designed with a bent at their tips (e.g., up to about 40°-45°). The effect of rotational oscillations on such structures could potentially be problematic; however, the present applicants report that these geometrical difficulties appeared to play no discernable role on the operational outcome. Indeed all of the tests that are reported herein were performed using 30° bent pipettes.

A test phase took place approximately 2 months into the trials. Approximately over 400 oocytes were injected. The cleavage rates varied from 70-90%. This is considered to be very successful. These results compare well against the conventional deployment of ICSI. At this stage, the experiments revealed the following:
The system 1000 renders an extremely successful piercing compared with non-mercury piezo-drilling. We showed 100% zona and membrane piercing capability with the system 1000, when piezo-drills without mercury could not work. Fluorinert (FC77, FC40) may replace mercury, but the present applicants are not aware of published data supporting the consequences.
The system 1000 compares well against Piezo-drill with mercury as well (about slightly higher than 80% cleavage rate) (Kimura 1995).
A noticeable difference between piezo-drill (with mercury) and the system 1000 appeared in the speed of lyses. If it occurs during the piercing with the system 1000 (which was rare), cytoplasm oozes slowly, as opposed to spilling out instantaneously in the piezo-drill case. This is an indication of minimally invasive piercing operation when the system 1000 is used.
Our first set of 35 live-born pups has arrived.

The system 1000 is well suited for intracytoplasmic sperm injection (ICSI). Methods of using the system 1000 include the deployment of rotational oscillations at the pipette tip as it engages with the cell membrane. Small angular amplitudes but high frequency of the oscillations may be used to facilitate the piercing through the membrane.

Techniques described herein for using the system 1000 offer many, clear advantages over state-of-the-art, including but not limited to: i) the system 1000 and the methods of using same substantially prevent the undesirable transverse oscillations at the tip of the pipette during piercing, ii) the system 1000 and the methods of using same result in comparatively high survival and cleavage rates with respect to the piezo-drill with mercury but without need for this toxic substance, and iii) the system 1000 may be substantially fully automated, and the training period needed for the operator need not necessarily be of long duration (e.g., in a range of weeks should be sufficient).

The experimental results reported here are from mouse ICSI implementations. However the system 1000 and/or the methods for using same have potential for broader usage. For example, the present applicants have tested the piercing capabilities of the system 1000 on Drosophila melanogaster and Drosophila willistoni. Prior penetration attempts by others failed very often due to breakage of the pipettes as the cell membrane is very hard, especially for Drosophila willistoni. Without going into details, the present applicants observed 100% successful piercing using the system 1000. This exercise is a representative example that the technology is portable to many other biological uses outside ICSI.

### Example 3

### Penetration of Mouse Oocytes

In this section and with the underlying study, the present applicants further describe the development and testing of a rotationally oscillating drill device and further develop a mouse ICSI technology without using toxic mercury. Using this new technology, the applicants obtained survival, fertilization, and blastocyst formation rates at least comparable to that of piezo-assisted ICSI.

The present applicants found it relatively easy to penetrate the mouse zona using a spiked micropipette. At least one aim of the present design was to build a drill that could rotationally isolate a pipette at a desired frequency and an angular amplitude to achieve two tasks: i) to enable separation of the sperm head and tail, and ii) to facilitate penetration of the oolemma. Because a perfectly straight pipette is impossible to be pulled even using a fully automatic puller, an eccentricity is expected. This feature substantially unavoidably causes some degree of whirring motion during rotational oscillation of the pipette holder. In order to avoid excessive lateral displacement in such cases, the pipette was oscillated with very small angular amplitudes (e.g., 1° peak-to-peak) and at frequencies which are higher than the sensitive natural frequencies of the pipette (e.g., our typical operating frequency is close to 500 Hz).

A microinjector system in accordance with the system 1000 described herein with reference to FIG. 10 was used. The pipette holder was placed in precision bearings, which were embedded within the body or housing. A flexible coupling that had a channel to accommodate the injection tubing was attached between the pipette holder and a micro-motor (which is typically a precision DC servo motor). The coupling transmits the angular motion from the motor to the pipette holder and also prevents axial misalignment. This DC micro-motor was energized via a linear amplifier (“Driver”). The control signal was generated by a digital controller. The reference signal for rotational oscillations of the pipette tip was harmonic A sin(2πft), where A (deg) is the amplitude of the oscillations, f (Hz) is the frequency of the oscillations, and t is time (seconds). A pure harmonic reference trajectory was purposely selected at a frequency so that unnecessary excitation of the natural vibration modes of the pipette was avoided. This trajectory is easy to generate and implement. Detailed technical information about the system 1000 can be found hereinabove.

In summary, the present applicants conceived at least two different modes of operation of the system 1000: i) low rotational amplitude, high frequency, for piercing the oolemma, and ii) high rotational amplitude and low frequency and impulsive behavior, for isolating the sperm head and tail.

KSOMAA medium (see, e.g., Biol. Reprod., Vol. 63, pp. 281-293 (2000) by J. D. Biggers et al. (Biggers 2000)) and FHM medium (see, e.g., Methods Enzymol., Vol. 225, pp. 153-164 (1993) by J. A. Lawitts and J. D. Biggers (Lawitts 1993)) and fetal bovine serum (FBS) were purchased from Specialty Media (Phillipsburg, N.J.). Polyvinyl pyrrolidone (PVP) was purchased from Western Medical Supply, Inc. (California). Pregnant mare serum gonadotrophin (PNSG) and human chorionic gonadotrophin (HCZB) were obtained from Sigma Chemical Co. (St. Louis, Mo.). Na-EGTA medium was Tris-buffered EGTA solution containing 10 mM Tris, 50 mM NaCl and 50 mM EGTA, pH 8.0.

Six to eight weeks old female and eight to ten weeks old male B6D2F1 mice were purchased from The Jackson Laboratory (Bar Harbor, Me.) and used as egg and sperm donors, respectively. Eight to ten weeks old CD1 mice from Charles River Laboratories, Inc. (Wilmington, Mass.) were used to produce vasectomized males and pseudopregnant recipients for embryo transfer. All mice were housed in individually ventilated plastic cages (BioZone, Inc., Fort Mill, S.C.) with bedding made from reclaimed wood pulp (Absorption Corporation, Bellingham, Wash.) in a specific pathogen free barrier facility with light cycle 14 h light and 10 h dark according to standard operating procedures of the University of California, Davis. Mice were fed ad libitum with food purchased from LabDiet (Richmond, Ind.), and were allowed free access to deionized, autoclaved water. Mouse euthanasia was carried out by CO₂ asphyxiation and cervical dislocation. The care, use, and disposition of all mice used in the study were reviewed and approved by the Institutional Animal Care and Use Committee of the University of California, Davis.

Sperm were collected from the caudal epididymids into HCZB or Na-EGTA medium, Sperm heads and tails were separated using three different techniques. In one, sperm heads were separated from tails by freezing 100 μl of sperm suspension in liquid nitrogen for 1 min followed by thawing in a water bath at 37° C. for 1 min. Using a second technique, a few piezo pulses (intensity 3-4, speed 3) were applied to the neck region after the sperm in 10% PVP in HCZB containing 0.1 mg/ml PVA was aspirated, tail first, into a flat tip pipette (diameter 7 μm). Using a third technique, sperm were aspirated into a spiked ICSI pipette, tail first, and placed at the midpiece near the sperm neck. Then a group of bi-directional rotational pulses was applied. The duration of each pulse was 6 ms and the frequency was 50 Hz. On average, the sperm tail was separated within less then a second. Sperm heads separated by freeze-thaw were kept on ice before use, and the sperm heads (5-10 in each group) prepared using ICSI pipettes were used immediately after preparation.

For purposes of ICSI performed using the system 1000 and methods described herein, straight human ICSI pipettes (diameter 7) from The Pipette Company-TPC (model LICR-ST) were used for ICSI performed on an inverted Nikon TE300 microscope (Nikon TE 300, Japan) with Nomarski differential interference contrast (DIC) optics at room temperature (FIG. 4). After washing in 10% PVP in HCZB, 5 to 10 sperm heads prepared using the methods described above were loaded into ICSI pipette with appropriate intervals of 10% PVP, and ICSI was performed in a drop of 10% FBS in HCZB (see Zygote, Vol. 5, pp. 111-116 (1997) by K. Suzuki and R. Yanagimachi (Suzuki 1997)).

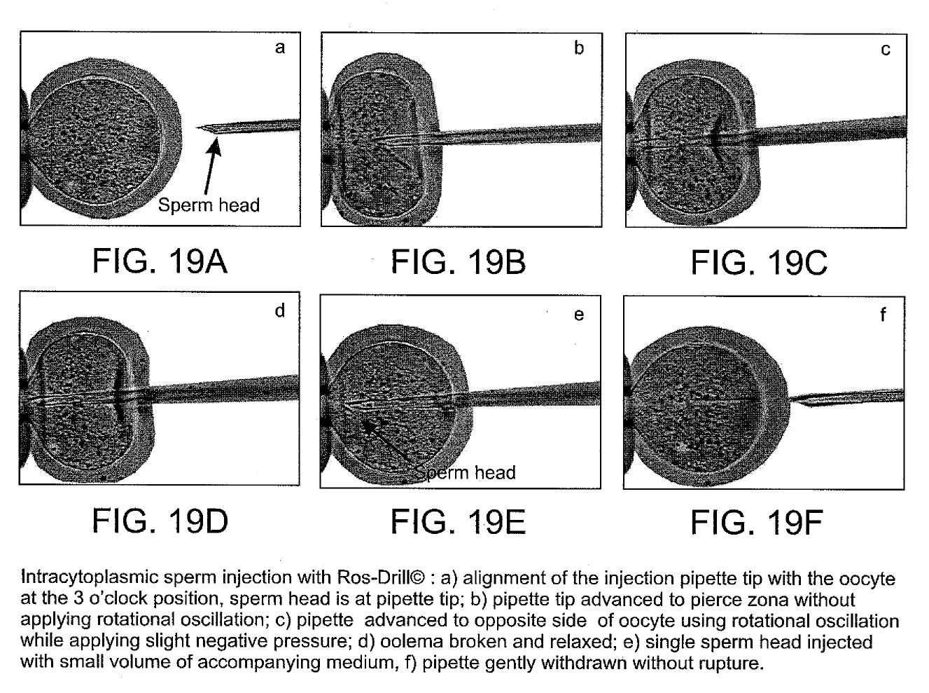

The zona could be readily and successfully penetrated using a spiked micropipette without the aid of rotational drilling. After zona penetration, the ICSI pipette was advanced against the oolemma towards the opposite pole of the oocyte, and a series of rotational oscillations (frequency 500 Hz, lowest amplitude) was applied until the oolemma was broken. The sperm head was then injected into the ooplasm with a minimum amount of accompanying medium. On average, it required 10-20 min to inject a group of 10-15 oocytes. The procedure is shown in FIG. 19.

For purposes of ICSI performed using piezo-assistance, the ICSI was performed with a PMM controller (Prime Tech, Ibaraki, Japan) using sperm in HCZB containing 10% FBS. The oocyte was held at the 9 o'clock position so that the metaphase II spindle was at either the 12 or 6 o'clock position. The injection pipette (diameter 7 μm, loaded with mercury) was advanced to penetrate the zona pellucida at the 3 o'clock position after applying several piezo-pulses (intensity 2-4, speed 3). The zona piece was expelled into the perivitelline space and the injection pipette was advanced against the oolemma to the opposite side of the oocyte's cortex. The oolemma was punctured by applying 1 weak piezo pulse (intensity 1-2, speed 1), and a sperm head was released into the ooplasm.

### Embryo Culture/Embryo Transfer:

Injected oocytes were washed and incubated in equilibrated KSOMAA medium (50 ml drops under mineral oil) humidified and warmed to 37.5° C. in 5% CO₂ and 95% air for 24-98 hours for 4 days. For in vitro experiments, embryos were graded for stage of development every 24 hours after ICSI. For in vivo experiments, blastocysts were transferred into the uterus (4-6 embryos each horn) of pseudopregnant CD-1 female mice (2.5 days post-coitum with vasectomized males) anesthetized with 2.5% Avertin. Recipients were kept warm on a heating pad until fully recovered from anesthesia. Before the recipients were conscious, 0.1 ml of 0.03 mg/ml Buprenex was injected subcutaneously to provide post-operative analgesia.

### Statistical Analysis:

Unordered and singly ordered contingency tables were analyzed with the Exact Fisher and the exact Kiruskal-Wallis test, respectively. Stratified 2×2 and 2×5 contingency tables were analyzed by the methods described in the manual of StatXact 8. All computations were done using StatXact 8 (Cytel Inc., Cambridge, Mass.).

### Results:

A comparison was made between, on the one hand, Piezo ICSI, and on the other hand, ICSI performed using the system 1000 and the methods discussed herein. Three replicates were done comparing the effects of injecting sperm heads into unfertilized ova using either Piezo-ICSI, where the sperm heads are isolated using either a piezo pulse, or via the presently disclosed ICSI methods, using sperm heads separated in by freeze-thaw in Na-EGTA medium. Each replicate was done by the same operator using the same sperm sample. The results from each of the three replicates can be considered to be a set of three stratified contingency tables, one set for each for the survival data, another set for the fertilization rate data, and another set for the embryo development data.

Ovum survival: A test to determine whether the odds-ratios of the three 2×2 contingency tables are the same shows they are homogeneous (P=0.288). The results in the three tables have therefore been combined (Table 2).

TABLE 2
The numbers of ova that survived the injection of sperm
heads using either Ros-Drill-ICSI or Piezo-ICSI.

| Method | Survivors | Non-survivors | Total |
|---|---|---|---|
| Ros-Drill-ICSI | 95 (81.9%) | 21 (18.1%) | 116 (100%) |
| Piezo-ICSI | 106 (95.5%) | 5 (4.5%) | 111 (100%) |
| Total | 201 (88.6%) | 26 (11.5%) | 227 (100%) |

Exact Fisher Test: P = 0.013..

The percentage of ova that survived when the sperm heads were injected using the system 1000 and the methods described herein was 81.9% (95/116), while the percentage that survived using Piezo ICSI was 95.5% (106/11). These two percentages are significantly different (Exact Fisher test: P=0.0015), showing that survival rate was slightly lower when Ros-Drill-ICSI was used.

Fertilization rate: A test to determine whether the odds-ratios of the three 2×2 contingency tables are the same shows they are homogeneous (P=1). The results in the three tables have therefore been combined (Table 3).

TABLE 3
The numbers of two-cell embryos that developed from the ova that
survived the injection of sperm heads using either the system
1000 and the ICSI methods described herein or Piezo-ICSI.

| | Developers | | |
|---|---|---|---|
| Method | (2-cells) | Non-developers | Total |
| Ros-Drill-ICSI | 83 (87.4%) | 12 (12.6%) | 95 (100%) |
| Piezo-ICSI | 103 (97.2%) | 3 (2.8%) | 106 (100%) |
| Total | 186 (92.5%) | 15 (7.5%) | 201 (100%) |

Exact Fisher Test: P = 0.013..

The percentage of injected ova that survived and developed into two-cell embryos (an estimate of the fertilization rate) observed using the system 1000 and the ICSI methods described herein was 87.4%, while the fertilization rate when Piezo-ICSI was used was 97.2%. The two percentages are significantly different (Exact Fisher test: P=0.032), showing that the fertilization rate was slightly lower when the system 1000 and the ICSI methods described herein was used.

Embryo development: The Cohran-Mantel-Haenszel test on singly ordered variables of the three stratified 2×5 contingency tables shows the distributions of the stages of development reach in each replicate are similar (P=0.405). The results have therefore been combined in Table 4.

TABLE 4
The development of embryos at various embryonic stages (2-cell through blastocysts)
after culture from the injection of sperm heads into unfertilized ova using either
the system 1000 and the ICSI methods described herein or Piezo-ICSI.

| | | | Non- | | | |
|---|---|---|---|---|---|---|
| | | | compacted | Compacted | | |
| Method | 2-cells | 3-4 cells | morulae | morulae | Blastocysts | Total |
| Ros-Drill-ICSI | 3 (3.6%) | 9 (10.8%) | 6 (7.2%) | 18 (21.7%) | 47 (56.6%) | 83 (100%) |
| Piezo-ICSI | 7 (6.8%) | 8 (7.8%) | 7 (6.8%) | 5 (4.9%) | 76 (73.8%) | 103 (100%) |
| Total | 10 (5.4%) | 17 (9.1%) | 13 (7.0%) | 23 (12.4%) | 123 66.1%) | 186 (100%) |

Exact Kruskal-Walk's Test: P = 0.069.

Comparison of the distributions of the stages of development reached when sperm were injected using the system 1000 and the ICSI methods described herein and Piezo-ICSI just fail to reach significance at the P=0.05 level (Kiruskal-Wallis test: P=0.069). Inspection of Table 4, however, shows that more compacted morulae developed into blastocysts when the Piezo-ICSI procedure (76/81; 93.8%) was used while fewer developed when the system 1000 and the ICSI methods described herein was used (47/65; 72.3%). These percentages are significantly different (P=0.0005).

### ICSI Using Sperm Heads Separated by Alternative Methods:

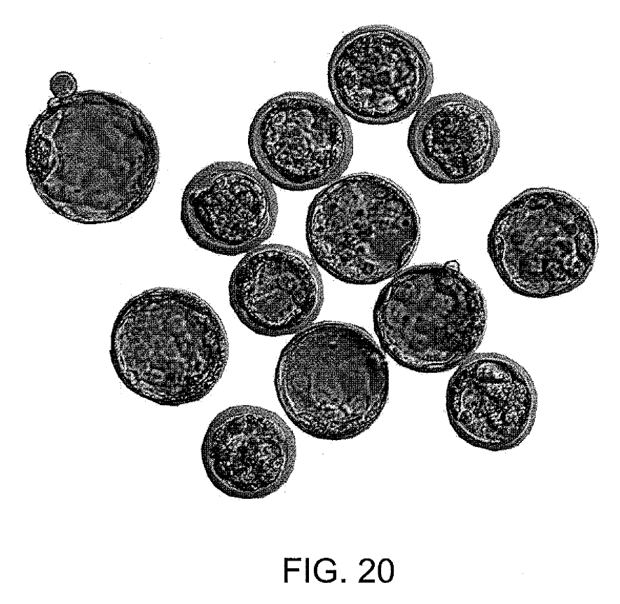

Three replicates were done in which groups of unfertilized ova were injected with sperm heads separated by freeze-thawing in HCZB medium. The percentage of ova that survived the injection ranged from 83-92% and were not significantly different (Exact Fisher test: P=0.556). When combined, the percentage survival rate was 87.5% (98/112). The percentage of survived ova that developed to the two-cell stage, a measure of the fertilization rate, ranged from 77-88% and were not significantly different (Exact Fisher test: P=0.488). When combined, the fertilization rate was 81.6% (80/98). The exact Kiruskal-Wallis test showed that the subsequent development to the blastocyst stage was very similar in all replicates. The data have been polled and are shown in Table 5. Overall, 47.5% (38/80) fertilized ova developed into blastocysts. Some of these blastocysts are shown in FIG. 20.

After trying different amplitudes and frequencies of the system 1000, it was found that the drill can also be used to separate the sperm heads of mouse spermatozoa (e.g., for the duration of 6 ms, execute an oscillatory motion corresponding to a frequency 50 Hz and a peak-to-peak rotational swing of about 4 degrees). The data are shown in Table 5. Overall, 64.5% (20/31) fertilized ova developed into blastocysts.

TABLE 5
The development of embryos at various embryonic stages (2-cell through blastocysts) after
culture from the injection of sperm heads isolated either by freeze-thaw in HCZB medium
or by the Ros-Drill into unfertilized ova using the Ros-Drill-ICSI procedure.

| | | | Non- | | | |
|---|---|---|---|---|---|---|
| | | | compacted | Compacted | | |
| Method | 2-cells | 3-4 cells | morulae | morulae | Blastocysts | Total |
| HCZB medium | 3 (3.8%) | 7 (8.8%) | 11 (13.8%) | 21 (26.3%) | 38 (47.5%) | 80 (100%) |
| Ros-Drill | 2 (6.5%) | 1 (3.2%) | 3 (9.7%) | 5 (16.1%) | 20 (64.5%) | 31 (100%) |

### Development of the Blastocysts to Term In Vivo:

Some of the blastocysts obtained by using sperm heads separated using freeze-thaw in Na-EGTA, freeze-thaw in HCZB and by the Piezo-Drill were transferred into the uterus of E2.5 pseudopregnant CD-1 and allowed to develop to term. The results are summarized in Table 6. The rates of pups born using the three methods are not significantly different (P=0.232).

TABLE 6
The numbers of pups born after transferring blastocysts produced
using sperm heads isolated by freeze-thaw in Na-EGTA, freeze-thaw
in HCZB and the Ros-Drill into surrogate mice.

| Method | No. transferred | No. pups |
|---|---|---|
| Freeze-thaw in | 47 | 9 (25%) |
| Na-EGTA | | |
| Freeze-thaw in | 36 | 9 (25%) |
| HCZB | | |
| Ros-Drill | 20 | 6 (30%) |

Exact Fisher Test: P = 0.232.

### Discussion

In recent years the use of metallic mercury in scientific equipment has been discouraged because of its toxicity. In some institutions, such as hospitals, there is a total ban on its use. The commonly used procedure for the injection of sperm heads into mouse unfertilized oocytes (ICSI) requires the use of a fine thread of mercury in the injection pipette. The injection apparatus and methods in this paper may be employed to avoid the use of mercury. The new equipment has been successfully used for ICSI in mice. The results show that the procedure is not as efficient as the Piezo protocol. Nevertheless the method is sufficiently effective for use in those laboratories where the use of mercury is banned.

Although the survival rate of injected ova and the fertilization rate is high (>80 percent in both) both rates are significantly less than the rates observed using Piezo-ICSI. In contrast, the rate of passage from the compacted morula to the blastocyst stage is considerably less using the new equipment than the rate seen using Piezo-ICSI. The reason for this delayed developmental effect is unknown.

The present applicants also have evidence that sperm heads can be separated from the midpiece using either freeze-thaw or the system 1000. Thus, in another mode of operation of the system 1000, mercury need not necessarily be used in the injection pipette to isolate sperm heads. So far the effects of freeze-thaw and the use of the system 1000 have not been associated with any developmental defects.

Although the present disclosure has been described with reference to exemplary embodiments of advantageous apparatus, systems, methods, and examples, the present disclosure is not limited by such exemplary embodiments. Rather, such exemplary embodiments are merely illustrative of potential implementations of the present disclosure. Indeed, the present disclosure expressly encompasses enhancements, modifications and/or variations on the disclosed embodiments that do not depart from either the spirit or the scope of the disclosed invention as set forth in the attached claims.

What is claimed is:

1. A microinjection device, comprising:
an injection element; and
a rotational motor;
wherein the injection element is rotatable about a longitudinal axis by the rotational motor, and the injection element is adapted to penetrate a target.

2. The microinjection device of claim 1, wherein the injection element includes a beveled distal end adapted to permit the injection element to penetrate a target.

3. The microinjection device of claim 1, wherein the injection element includes a spiked distal end adapted to permit the injection element to penetrate a target.

4. The microinjection device of claim 1, wherein the injection element is a micropipette, cannula, or needle.

5. The microinjection device of claim 1, wherein the rotational motor is adapted to rotate the injection element alternately clockwise and counterclockwise about the longitudinal axis.

6. The microinjection device of claim 5, wherein the rotational motor is adapted to rotationally oscillate the injection element about the longitudinal axis.

7. The microinjection device of claim 1, further comprising an injection element holder, wherein the injection element holder couples the injection element to the rotational motor.

8. The microinjection device of claim 7, wherein the injection element holder provides injectable materials to the injection element.

9. The microinjection device of claim 1, wherein the rotational motor is a micromotor.

10. The microinjection device of claim 9, wherein the micromotor rotates the injection element in alternate directions about the longitudinal axis within a range of angular motion of between about 0.5 degrees and about 10 degrees peak-to-peak.

11. The microinjection device of claim 9, wherein the micromotor rotates the injection element in alternate directions about the longitudinal axis within a range of angular motion of between about 0.5 degrees and about 2 degrees peak-to-peak.

12. The microinjection device of claim 9, wherein the micromotor oscillates the injection element about the longitudinal axis with a frequency of about 10 to about 500 cycles per second.

13. The microinjection device of claim 1, further comprising means for manipulating the injection element.

14. The microinjection device of claim 13, wherein the means for manipulating the injection element include a micromanipulator.

15. The microinjection device of claim 1, wherein the target is selected from the group consisting of a cell, cell nucleus, embryo, ovum, oocyte, and zygote.

16. A microinjection system comprising:
the microinjection device of claim 1; and
a control unit;
wherein the control unit is adapted to control a rotational amplitude and a frequency of oscillation of the injection element.

17. The microinjection system of claim 16, wherein the microinjection device further includes an injection element positioner for translationally moving the injection element with respect to the target.

18. The microinjection system of claim 16, further comprising means for manipulating the target during an injection procedure.

19. The microinjection system of claim 18, wherein the means for manipulating the target includes a holding pipette, and a micromanipulator coupled to the holding pipette, the micromanipulator being adapted to manipulate the holding pipette during an injection procedure for purposes of at least one of stabilizing the target, and moving the target.

20. A method for penetrating a target to facilitate injecting material therein, the method comprising:
providing the material to an injection element;
contacting the target with a distal end of the injection element;
rotating the injection element about a longitudinal axis to form a hole in the target; and
penetrating the target with the injection element via the hole formed in the target.

21. The method of claim 20, wherein the rotating step includes rotating the injection element alternately clockwise and counterclockwise about the longitudinal axis in an oscillatory manner to form the hole in the target.

22. The method of claim 21, wherein the step of rotating the injection element alternately clockwise and counterclockwise about the longitudinal axis in an oscillatory manner includes causing the injection element to oscillate within a range of angular motion of between about 0.5 degrees and about 10 degrees peak-to-peak.

23. The method of claim 21, wherein the step of rotating the injection element alternately clockwise and counterclockwise about the longitudinal axis in an oscillatory manner includes causing the injection element to oscillate within a range of angular motion of between about 0.5 degrees and about 2 degrees peak-to-peak.

24. The method of claim 20, wherein the step of contacting the target with a distal end of the injection element includes one or both of translationally moving the injection element toward the target and translationally moving the target toward the injection element.

25. The method of claim 20, further comprising expelling the material into the penetrated target.

26. The method of claim 20, wherein the longitudinal axis is defined by a longitudinal extent of the injection element.

27. A method for performing intra-cytoplasmic sperm injection comprising:
providing a solution comprising sperm to an injection element;
contacting an oocyte with a distal end of the injection element;
rotating the injection element alternately clockwise and counterclockwise about a longitudinal axis to form a hole in the oocyte;
penetrating the oocyte with the distal end of the injection element via the hole formed in the oocyte; and
expelling the solution comprising sperm into the penetrated oocyte.

28. The method of claim 27, wherein the step of rotating the injection element alternately clockwise and counterclockwise about a longitudinal axis includes causing the injection element to oscillate within a range of angular motion of between about 0.5 degrees and about 10 degrees peak-to-peak.

29. The method of claim 27, wherein the step of rotating the injection element alternately clockwise and counterclockwise about a longitudinal axis includes causing the injection element to oscillate within a range of angular motion of between about 0.5 degrees and about 2 degrees peak-to-peak.

30. The method of claim 27, wherein the step of contacting an oocyte with a distal end of the injection element includes deflecting inward a cell membrane of the oocyte.
